(19) United States
(12) Patent Application Publication
(10) Pub. No.: US 2020/0229435 A1
(43) Pub. Date: Jul. 23, 2020

(54) ANTIMICROBIAL MIXTURE CONTAINING 4-(3-ETHOXY-4-HYDROXYPHENYL)BUTAN-2-ONE AND AN AMMONIUM COMPOUND, AND COSMETIC COMPOSITION CONTAINING SAME

(71) Applicant: L'OREAL, Paris (FR)

(72) Inventors: Gael MALET, Chevilly Larue (FR); Sylvie CUPFERMAN, Chevilly Larue (FR); Florence MENARD-SZCZEBARA, Chevilly Larue (FR)

(21) Appl. No.: 16/630,058

(22) PCT Filed: Jun. 27, 2018
(86) PCT No.: PCT/EP2018/067300

§ 371 Date: Jan. 10, 2020

(30) Foreign Application Priority Data
Aug. 18, 2017 (FR) 1757744
Aug. 18, 2017 (FR) 1757746

(51) Int. Cl. A01N 35/02; A01N 33/04; A61K 8/35; A61K 8/41

(52) U.S. Cl. CPC A01N 35/02; A61K 8/416; A61K 8/35; A01N 33/04

(57) ABSTRACT

The invention relates to an antimicrobial mixture containing 4-(3-ethoxy-4-hydroxy-phenyl)butan-2-one and an ammonium compound chosen from a fatty-chain C₂₀ to C₂₄ tri-methylammonium salt and a (C₈-C₂₈)alkyldimethylbenzylammonium salt, and also to a cosmetic composition containing such a mixture. Use into caring for, making up and cleansing keratin materials.

A subject of the present invention is an antibacterial mixture containing 4-(3-ethoxy-4-hydroxyphenyl)butan-2-one and a particular ammonium compound, and also after a cosmetic composition containing such a mixture.

4-(3-Ethoxy-4-hydroxyphenyl)butan-2-one (ketone compound) is a useful substance as a preserving agent for cosmetic compositions, for protecting the compositions against microbial contamination, as described in patent application WO 2011/039445.

However, it is desirable to be able to incorporate said ketone compound in reduced concentration in compositions, especially cosmetic or dermatological compositions, while at the same time maintaining good antimicrobial conservation performance. Combinations of the ketone compound with other preserving agents that have good antimicrobial efficacy are thus sought for this purpose.

The inventors have discovered, unexpectedly, that the combination of 4-(3-ethoxy-4-hydroxyphenyl)butan-2-one with an ammonium compound chosen from a fatty-chain alkyltrimethylammonium salt and an alkyldimethylbenzylammonium salt as defined below, in particular behenyltrimethylammonium chloride or an alkyldimethylbenzylammonium chloride, makes it possible to obtain an antimicrobial mixture with synergistic antimicrobial activity, in particular at least on moulds, especially on Aspergillus niger.

The results of the examples described below show the synergistic antimicrobial activity obtained with the minimum inhibitory concentration (MIC) measurements taken with several mixtures. The antimicrobial activity is considered as being synergistic when the antimicrobial mixture makes it possible to obtain a percentage of strain growth of less than or equal to 25%, or even less than or equal to 20%.

The combination of 4-(3-ethoxy-4-hydroxyphenyl)butan-2-one with a particular fatty-chain alkyltrimethylammonium salt as defined below, in particular behenyltrimethylammonium chloride, makes it possible to obtain an antimicrobial mixture with synergistic antimicrobial activity, in particular on moulds, especially on Aspergillus niger, on yeasts, in particular on Candida albicans, and on gram-negative bacteria, especially on Pseudomonas aeruginosa.

The combination of 4-(3-ethoxy-4-hydroxyphenyl)butan-2-one with an alkyldimethylammonium salt as defined below, in particular an alkyldimethylbenzylammonium chloride, makes it possible to obtain an antimicrobial mixture with synergistic antimicrobial activity, in particular on moulds, especially on Aspergillus niger.

More precisely, a subject of the invention is an antimicrobial mixture comprising, or constituted by (or consisting of), 4-(3-ethoxy-4-hydroxyphenyl)butan-2-one and an ammonium compound chosen from:

i) an alkyltrimethylammonium salt of formula R—N⁺(CH₃)₃ X⁻ (I) in which R denotes an alkyl group containing from 20 to 24 carbon atoms, X⁻ denotes a halide ion, in particular chloride or bromide, or a methosulfate ion;
ii) an alkyldimethylbenzylammonium salt of formula (II):

C₆H₅—CH₂—N⁺(CH₃)₂R X⁻ (II)

in which R denotes an alkyl radical containing from 8 to 18 carbon atoms,
X⁻ denotes a halide anion such as chloride or bromide.

A subject of the invention is also a composition, especially a cosmetic composition, comprising, in a physiologically acceptable medium, said mixture described previously.

A further subject of the invention is a process for the non-therapeutic cosmetic treatment of keratin materials, comprising the application to the keratin materials of a composition as described previously. The process may be a cosmetic process for caring for or making up or cleansing keratin materials.

A subject of the invention is also a process for conserving a composition comprising a physiologically acceptable medium, in particular a cosmetic or dermatological composition, characterized in that it consists in incorporating into said composition an antimicrobial mixture as described previously.

A subject of the invention is also the use of the antimicrobial mixture described previously for conserving a composition comprising a physiologically acceptable medium.

4-(3-Ethoxy-4-hydroxyphenyl)butan-2-one is a compound of formula:

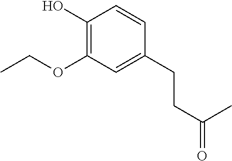

According to a first embodiment, a subject of the invention is an antimicrobial mixture comprising, or constituted by (or consisting of), 4-(3-ethoxy-4-hydroxyphenyl)butan-2-one and an alkyltrimethylammonium salt of formula R—N⁺(CH₃)₃ X⁻ (I) in which R denotes an alkyl group containing from 20 to 24 carbon atoms, X⁻ denotes a halide ion, in particular chloride or bromide, or a methosulfate ion.

For the alkyltrimethylammonium salt of formula (I) described previously, R is preferably a behenyl group.

Preferably, X⁻ is a chloride anion.

Advantageously, the alkyltrimethylammonium salt (I) is behenyltrimethylammonium chloride (INCl name: behentrimonium chloride).

Behenyltrimethylammonium chloride may be used as a mixture at 79% by weight with 18% of isopropyl alcohol and 3% of water sold under the name Genamin KDMF by the company Clariant.

Advantageously, 4-(3-ethoxy-4-hydroxyphenyl)butan-2-one and the trimethylammonium salt (I) are present in said mixture in a content such that the 4-(3-ethoxy-4-hydroxyphenyl)butan-2-one/salt (I) (preferably behenyltrimethylammonium chloride) weight ratio ranges from 0.08 to 80, preferably from 0.45 to 70 and preferentially from 1.5 to 50.

The antimicrobial mixture according to the invention has synergistic antimicrobial activity, in particular on moulds, especially on Aspergillus niger, on yeasts, in particular on Candida albicans, and on gram-negative bacteria, in particular on Pseudomonas aeruginosa.

Preferentially, the antimicrobial mixture according to the invention contains behenyltrimethylammonium chloride.

The antimicrobial mixture may have a 4-(3-ethoxy-4-hydroxyphenyl)butan-2-one/salt (I) (preferably behenyltrimethylammonium chloride) weight ratio ranging from 10 to 60, preferably ranging from 10 to 55 and preferentially ranging from 15 to 40, and better still ranging from 20 to 30. Such a mixture has good antimicrobial activity on yeasts, especially on Candida albicans.

The antimicrobial mixture may have a 4-(3-ethoxy-4-hydroxyphenyl)butan-2-one/salt (I) (preferably behenyltrimethylammonium chloride) weight ratio ranging from 0.08 to 5, preferably ranging from 0.45 to 4.5, preferentially ranging from 0.45 to 3, better still from 0.4 to 2.5, and preferentially ranging from 1.5 to 3. Such a mixture has good antimicrobial activity on moulds, especially on Aspergillus niger.

The antimicrobial mixture may have a 4-(3-ethoxy-4-hydroxyphenyl)butan-2-one/salt (I) (preferably behenyltrimethylammonium chloride) weight ratio ranging from 3 to 80, preferably ranging from 3 to 70, preferentially ranging from 3 to 50 and better still ranging from 3 to 45. Such a mixture has good antimicrobial activity on gram-negative bacteria, especially on Pseudomonas aeruginosa.

According to a second embodiment, a subject of the invention is an antimicrobial mixture comprising, or constituted by (or consisting of), 4-(3-ethoxy-4-hydroxyphenyl)butan-2-one and an alkyldimethylbenzylammonium salt of formula (II):

C₆H₅—CH₂—N⁺(CH₃)₂R X⁻ (II)

in which R denotes an alkyl radical containing from 8 to 18 carbon atoms,
X⁻ denotes a halide anion such as chloride or bromide.

For the alkyldimethylbenzylammonium salt of formula (II) described previously, R is preferably an alkyl group comprising from 10 to 16 carbon atoms and preferentially from 12 to 14 carbon atoms.

Preferably, X⁻ is a chloride anion.

Use is preferably made of an alkyldimethylbenzylammonium chloride (INCl name: benzalkonium chloride).

Advantageously, the alkyldimethylbenzylammonium salt is a mixture of lauryldimethylbenzylammonium chloride (compound IIa; R=C12 alkyl) and of myristyldimethylbenzylammonium chloride (compound IIb; R=C14 alkyl).

Use is preferably made of a mixture chosen from:
a) a mixture comprising from 60% to 80% of compound IIa and 20% to 40% of compound IIb
b) a mixture comprising from 65% to 75% of compound IIa and 25% to 35% of compound IIb
c) a mixture comprising from 68% to 72% of compound IIa and 28% to 32% of compound IIb
d) a mixture comprising 70% of compound IIa and 30% of compound IIb.

The percentages indicated are molar percentages relative to the total number of moles of said mixture.

Advantageously, 4-(3-ethoxy-4-hydroxyphenyl)butan-2-one and the alkyldimethylbenzylammonium salt (II) are present in said mixture in contents such that the 4-(3-ethoxy-4-hydroxyphenyl)butan-2-one/salt (II) (preferably an alkyldimethylbenzylammonium chloride) weight ratio ranges from 5 to 120, preferably from 5 to 100, preferentially from 5 to 85, more preferentially from 30 to 85 and in particular from 35 to 85.

The antimicrobial mixture according to the invention has synergistic antimicrobial activity, in particular on moulds, especially on Aspergillus niger.

Preferentially, the antimicrobial mixture according to the invention contains an alkyldimethylbenzylammonium chloride.

A subject of the invention is also a composition comprising, in a physiologically acceptable medium, the antimicrobial mixture described previously.

The term “physiologically acceptable medium” means a medium that is compatible with human keratin materials such as the skin, the scalp, the hair and the nails. Said medium may comprise one or more additional ingredients other than the ketone compound and the ammonium compound described previously.

The compound 4-(3-ethoxy-4-hydroxyphenyl)butan-2-one may be present in the composition according to the invention at an amount ranging from 0.01% to 5% by weight relative to the total weight of the composition, preferably ranging from 0.01% to 3% by weight, preferentially ranging from 0.01% to 2.5% by weight and more preferentially ranging from 0.01% to 2% by weight.

The composition may comprise at least one additional ingredient chosen from water, oils, polyols containing from 2 to 10 carbon atoms, gelling agents, surfactants, film-forming polymers, dyestuffs, fragrances, fillers, UV-screening agents, plant extracts, cosmetic and dermatological active agents, and salts.

The composition according to the invention may comprise an aqueous phase.

The composition may comprise water, which may be present at a content ranging from 5% to 90% by weight relative to the total weight of the composition, and preferably ranging from 35% to 75% by weight.

The composition may also comprise a polyol that is water-miscible at room temperature (25° C.), especially chosen from polyols especially containing from 2 to 10 carbon atoms, preferably containing from 2 to 6 carbon atoms, such as glycerol, propylene glycol, 1,3-propanediol, butylene glycol, pentylene glycol, hexylene glycol, dipropylene glycol, diethylene glycol or diglycerol. Advantageously, the composition according to the invention comprises 1,3-propanediol, especially in a content ranging from 0.1% to 20% by weight, preferably ranging from 0.1% to 10% by weight and preferentially ranging from 0.5% to 5% by weight, relative to the total weight of the composition.

The compositions according to the invention may be in the form of oil-in-water (O/W) emulsions, water-in-oil (W/O) emulsions or multiple emulsions (triple: W/O/W or O/W/O), oily solutions, oily gels, aqueous solutions, aqueous gels, solid compositions. These compositions are prepared according to the usual methods.

The compositions according to the invention may be more or less fluid and may have the appearance of a white or coloured cream, an ointment, a milk, a lotion, a serum, a paste or a foam. They may be optionally applied to the skin in aerosol form. They may also be in solid form, for example in the form of a stick or a compact powder.

The composition according to the invention may especially be in the form of:
a makeup product, especially for making up the skin of the face, the body, or the lips or the eyelashes;
an aftershave gel or lotion; a shaving product;
a deodorant (stick, roll-on or aerosol);
a hair-removing cream;
a body hygiene composition such as a shower gel or a shampoo;
a pharmaceutical composition;
a solid composition such as a soap or a cleansing bar;
an aerosol composition also comprising a pressurized propellant;
a hairsetting lotion, a hair-styling cream or gel, a dye composition, a permanent-waving composition, a lotion or a gel for combating hair loss, or a hair conditioner;
a composition for caring for or cleansing the skin.

A subject of the invention is also a process for preparing a composition, especially a cosmetic or dermatological composition, comprising a step of mixing 4-(3-ethoxy-4-hydroxyphenyl)butan-2-one, the ammonium compound described previously, and one or more additional ingredients, especially cosmetic or dermatological ingredients, such as those described previously.

The invention is illustrated in greater detail in the example that follows. The amounts of the ingredients are expressed as weight percentages.

### EXAMPLE 1: DETERMINATION OF THE SYNERGISTIC ANTIMICROBIAL ACTIVITY AS MIC

The demonstration of a synergistic antimicrobial activity effect with a mixture of 4-(3-ethoxy4-hydroxyphenyl)butan-2-one (referred to as substance A) and of ammonium compound (referred to as substance B) is performed by calculating the synergy index (or FIC index).

### Formulation

FIC Index=(MICa with B/MICa)+(MICb with A/MICb)

with:
MICa with B: minimum concentration of product A in the combination A+B which makes it possible to obtain an inhibitory effect
MICb with A: minimum concentration of product B in the combination A+B which makes it possible to obtain the inhibitory effect.
MICa: minimum inhibitory concentration of product A alone.
MICb: minimum inhibitory concentration of product B alone.

This formula was described for the first time in the article by F. C. Kull, P. C. Eisman, H. D. Sylwestrowka, and R. L. Mayer, Applied Microbiology 9:538-541, 1961.

For each compound tested alone, the MIC is considered as the first concentration which makes it possible to obtain a microbial growth percentage of less than or equal to 25%.

As regards the combinations tested, MICa with b and MICb with a are the respective concentrations of A and of B in the combinations which make it possible to obtain a microbial growth percentage of less than or equal to 25%.

### Interpretation of the FIC Index:

When the FIC index value is less than or equal to 1, it is considered that the combination of test compounds has a synergistic effect.

The results obtained are summarized in the following tables.

The combination of compounds A and B was tested on the following strains or a part of these strains: Aspergillus niger, Candida albicans, Pseudomonas aeruginosa.

The microbial strain Aspergillus niger ATCC 6275, and a double-concentration Sabouraud broth liquid culture medium supplemented with polyoxyethylenated (20 OE) sorbitan monopalmitate (Tween 40 from Croda) and Phytagel© BioReagent were used.

The microbial strain Candida albicans ATCC 10231 and a double-concentration Sabouraud broth liquid culture medium were used.

The microbial strain Pseudomonas aeruginosa ATCC 9027 and a double-concentration nutritive broth liquid culture medium were used.

A 96-well microplate and an incubation time of 32.5° C. are used.

The incubation time of the microplate is:
from 24 hours to 48 hours aerobically for Aspergillus niger;
from 18 to 24 hours aerobically for Candida albicans and Pseudomonas aeruginosa.

### Tests

For each compound:

A=4-(3-ethoxy-4-hydroxyphenyl)-2-butanone compound

B=behenyltrimethylammonium chloride (as a mixture at 79% by weight with 18% of isopropyl alcohol and 3% of water sold under the name Genamin KDMF by the company Clariant*)
It is considered that the isopropyl alcohol present has no influence on the MIC results obtained.

A 10% (weight/volume) stock solution was prepared by mixing 1 g of compound in 9 ml of aqueous 1% agar solution. Successive dilutions were made with the 1% agar solution.
Tests of compounds A and B alone

50 μL of each of the daughter solutions obtained containing compound A or B are added to the microplate wells. 100 μL of Sabouraud liquid nutrient broth seeded with the strain Aspergillus niger and 50 μL of aqueous 1% agar solution are also added thereto.
Tests of compounds A and B as a mixture

50 μL of each of the daughter solutions obtained containing compound A and 50 μL of each of the daughter solutions obtained containing compound B are added to the microplate wells. 100 μL of Sabouraud liquid nutrient broth seeded at double concentration with the strain Aspergillus niger are also added thereto.

### Microbial Growth Control

A positive microbial growth control was also prepared. The positive microbial growth control corresponds to a mixture of 100 μL of aqueous 1% agar solution with 100 μL of Sabouraud liquid nutrient broth seeded at double concentration with the strain Aspergillus niger in the absence of compounds A and B.

### Absorbance Control of Compounds A and B Alone

An absorbance control was performed in parallel on compounds A and B alone. This control corresponds to 100 μL of double concentration sterile Sabouraud liquid nutrient broth+100 μL of double concentration compound A or B.

In the three cases (absorbance control, growth control and test), the final volume present in each of the microplate wells is 200 μL.

In the two cases (test and control), the inoculum represents the concentration of the strain Aspergillus niger present in the final volume of the wells (200 μL) and is between 2 and 6×10⁵ cfu/ml of Aspergillus niger.

The minimum inhibitory concentration (MIC) of each compound A and B alone and in combination was determined in a known manner by means of optical density measurements at a wavelength of 620 nm.

The test as described above (tests, absorbance control and growth control) was performed again to test the combination A+B on the strains Candida albicans and Pseudomonas aeruginosa.

The following results were obtained with

B1=behenyltrimethylammonium chloride (as a mixture at 79% by weight with 18% of isopropyl alcohol and 3% of water sold under the name Genamin KDMF by the company Clariant*)
It is considered that the isopropyl alcohol present has no influence on the MIC results obtained.
   Candida albicans

| | Concentrations tested (in weight %) | | | | |
|---|---|---|---|---|---|
| | 0 A | 0.025 A | 0.05 A | 0.1 A | 0.2 A |
| 0 B1 | | 73 | 73 | 49 | 9 |
| 0.002 B1 | 50 | 13 | 8 | 14 | 1 |
| | | (FIC 0.63) | (FIC 0.75) | (FIC 1) | |
| 0.00395 B1 | 1 | 0 | 0 | 1 | 1 |

| % MIC | % MIC | MIC of each compound | | | |
|---|---|---|---|---|---|
| of A | of B1 | as a mixture | | | |
| alone | alone | A % | B1 % | FIC Index | Ratio A/B1 |
| 0.2 | 0.00395 | 0.025 | 0.002 | 0.63 | 12.5 |

The results obtained show synergistic inhibitory activity for the mixtures:

i) 0.025% of A and 0.002% of B1, i.e. ratio A/B1=12.5

ii) 0.05% of A and 0.002% of B1, i.e. ratio A/B1=25

iii) 0.1% of A and 0.002% of B1, i.e. ratio A/B1=50

Aspergillus niger

| Concentrations | | | | | | |
|---|---|---|---|---|---|---|
| tested | | | | | | |
| (in weight %) | 0 A | 0.025 A | 0.05 A | 0.1 A | 0.2 A | 0.4 A |
| 0 B1 | | 150 | 113 | 112 | 75 | 5 |
| 0.049 B1 | 186 | 138 | 178 | −8 | −7 | −15 |
| | | | | (FIC 0.31) | (FIC 0.56) | |
| 0.1 B1 | 155 | 139 | 114 | 18 | −7 | 8 |
| | | | | (FIC 0.37) | (FIC 0.62) | |
| 0.2 B1 | 78 | 131 | 100 | 15 | −21 | 9 |
| | | | | (FIC 0.49) | (FIC 0.74) | |
| 0.4 B1 | 34 | 66 | −14 | 19 | −14 | −10 |
| | | | (FIC 0.60) | (FIC 0.73) | (FIC 0.98) | |
| 0.79 B1 | 8 | 20 | 26 | −35 | −20 | −50 |

| | | MIC of each | | | |
|---|---|---|---|---|---|
| % MIC | % MIC | compound as a mixture | | | |
| of A alone | of B1 alone | A % | B1 % | FIC Index | Ratio A/B1 |
| 0.4 | 0.79 | 0.1 | 0.049 | 0.31 | 2 |

The results obtained show synergistic inhibitory activity for the mixtures:

i) 0.05% of A and 0.4% of B1, i.e. ratio A/B1=0.125

ii) 0.1% of A and 0.4% of B1, i.e. ratio A/B1=0.25

iii) 0.1% of A and 0.2% of B1, i.e. ratio A/B1=0.5

iv) 0.1% of A and 0.1% of B1, i.e. ratio A/B1=1

v) 0.1% of A and 0.049% of B1, i.e. ratio A/B1=2

vi) 0.2% of A and 0.4% of B1, i.e. ratio A/B1=0.5

vii) 0.2% of A and 0.2% of B1, i.e. ratio A/B1=1

viii) 0.2% of A and 0.1% of B1, i.e. ratio A/B1=2

ix) 0.2% of A and 0.049% of B1, i.e. ratio A/B1=4

Pseudomonas aeruginosa

| concentrations | | | | | | |
|---|---|---|---|---|---|---|
| tested | | | | | | |
| (in weight %) | 0 A | 0.0625 A | 0.125 A | 0.25 A | 0.5 A | 1 A |
| 0 B1 | | 114 | 59 | 65 | 41 | 15 |
| 0.001975 B1 | 68 | 85 | 64 | 49 | 45 | 49 |
| 0.00395 B1 | 93 | 65 | 75 | 52 | 51 | 21 |
| 0.0079 B1 | 36 | 35 | 31 | 25 | 18 | 8 |
| | | | | | (FIC 0.75) | |
| 0.0158 B1 | 30 | 5 | 0 | −1 | 0 | 40 |
| | | (FIC 0.56) | (FIC 0.63) | (FIC 0.75) | (FIC 1) | |
| 0.0316 B1 | 1 | −3 | −2 | 0 | 4 | −34 |

| | | MIC of each | | | |
|---|---|---|---|---|---|
| % MIC | % MIC | compound as a mixture | | | |
| of A alone | of B1 alone | A % | B1 % | FIC Index | Ratio A/B1 |
| 1 | 0.0316 | 0.0625 | 0.0158 | 0.56 | 3.95 |

The results obtained show synergistic inhibitory activity for the mixtures:

i) 0.0625% of A and 0.0158% of B1, i.e. ratio A/B1=3.95

ii) 0.125% of A and 0.0158% of B1, i.e. ratio A/B1=7.91

iii) 0.25% of A and 0.0158% of B1, i.e. ratio A/B1=15.82

iv) 0.5% of A and 0.0158% of B1, i.e. ratio A/B1=31.64

v) 0.5% of A and 0.0079% of B1, i.e. ratio A/B1=63.29

### EXAMPLE 2: DETERMINATION OF THE SYNERGISTIC ANTIMICROBIAL ACTIVITY AS MIC

The demonstration of a synergistic antimicrobial activity effect with a mixture of 4-(3-ethoxy-4-hydroxyphenyl)butan-2-one (referred to as substance A) and of (C₁₂-C₁₄)alkyldimethylbenzylammonium chloride (referred to as substance B2) is performed according to the protocol described in Example 1, using the strain Aspergillus niger.

B2=(C₁₂-C₁₄)alkyldimethylbenzylammonium chloride=mixture of 70 mol % of benzyldimethyldodecylammonium chloride and 30 mol % of benzyldimethyltetradecylammonium chloride (sold under the name Benzalkonium chloride product No. 12060 by the company Sigma-Aldrich)

The following results were obtained:

Aspergillus niger

| Concentrations | | | | | | |
|---|---|---|---|---|---|---|
| tested | | | | | | |
| (in weight %) | 0 A | 0.025 A | 0.05 A | 0.1 A | 0.2 A | 0.4 A |
| 0 B2 | | 212 | 92 | 68 | 29 | 17 |
| 0.0025 B2 | 194 | 159 | 92 | 2 | 2 | 1 |
| | | | | (FIC 0.5) | (FIC 0.75) | |
| 0.005 B2 | 194 | 164 | 2 | 2 | 2 | 2 |
| | | | (FIC 0.63) | (FIC 0.75) | (FIC 1) | |
| 0.01 B2 | 2 | 2 | 2 | 2 | 2 | 2 |

| | | MIC of each | | | |
|---|---|---|---|---|---|
| % MIC | % MIC | compound as a mixture | | | |
| of A alone | of B2 alone | A % | B2 % | FIC Index | Ratio A/B2 |
| 0.4 | 0.01 | 0.05 | 0.005 | 0.63 | 10 |

The results obtained show synergistic inhibitory activity for the mixtures:

i) 0.05% of A and 0.005% of B2, i.e. ratio A/B2=10

ii) 0.1% of A and 0.005% of B2, i.e. ratio A/B2=20

iii) 0.1% of A and 0.0025% of B2, i.e. ratio A/B2=40

iv) 0.2% of A and 0.005% of B2, i.e. ratio A/B2=40

v) 0.2% of A and 0.0025% of B2, i.e. ratio A/B2=80

1. An antimicrobial mixture comprising 4-(3-ethoxy-4-hydroxyphenyl)butan-2-one and an ammonium compound chosen from:
i) an alkyltrimethylammonium salt of formula R—N⁺(CH₃)₃ X⁻ (I) in which R denotes an alkyl group containing from 20 to 24 carbon atoms, X⁻ denotes a halide ion or a methosulfate ion;
ii)

2. The antimicrobial mixture according to claim 1, wherein the ammonium compound is the alkyltrimethylammonium salt of formula (I).

3. The antimicrobial mixture according to claim 2, wherein for the trimethyl ammonium salt of formula (I), R is a behenyl group.

4. The antimicrobial mixture according to claim 2, wherein the alkyltrimethylammonium salt (I) is behenyltrimethylammonium chloride.

5. The antimicrobial mixture according to claim 2, which comprises 4-(3-ethoxy-4-hydroxyphenyl)butan-2-one and the alkyltrimethylammonium salt of formula (I) in amounts such that the 4-(3-ethoxy-4-hydroxyphenyl) butan-2-one/salt (I) weight ratio ranges from 0.08 to 80.

6. The antimicrobial mixture according to claim 2, which has a 4-(3-ethoxy-4-hydroxyphenyl)butan-2-one/alkyltrimethylammonium salt (I) weight ratio ranging from 10 to 60.

7. The antimicrobial mixture according to claim 2, which has a 4-(3-ethoxy-4-hydroxyphenyl)butan-2-one/alkyltrimethylammonium salt (I) weight ratio ranging from 0.08 to 5.

8. The antimicrobial mixture according to claim 2, which has a 4-(3-ethoxy-4-hydroxyphenyl)butan-2-one/alkyltrimethylammonium salt (I) weight ratio ranging from 3 to 80.

9. The antimicrobial mixture according to claim 1, wherein the ammonium compound is the alkyldimethylbenzylammonium salt of formula (II).

10. The antimicrobial mixture according to claim 9, wherein, for the alkyldimethylbenzylammonium salt of formula (II), R is an alkyl group comprising from 10 to 16 carbon atoms.

11. The antimicrobial mixture according to claim 9, wherein the alkyldimethylbenzylammonium salt (II) is an alkyldimethylbenzylammonium chloride.

12. The antimicrobial mixture according to claim 9, wherein the alkyldimethylbenzylammonium salt (II) is a mixture of lauryldimethylbenzylammonium chloride and myristyldimethylbenzylammonium chloride.

13. The antimicrobial mixture according to claim 12, wherein the alkyldimethylbenzylammonium salt (II) is chosen from the following mixtures:
a) a mixture comprising from 60 mol % to 80 mol % of lauryldimethylbenzylammonium chloride and 20 mol % to 40 mol % of myristyldimethylbenzylammonium chloride
b) a mixture comprising from 65 mol % to 75 mol % of lauryldimethylbenzylammonium chloride and 25 mol % to 35 mol % of myristyldimethylbenzylammonium chloride
c) a mixture comprising from 68 mol % to 72 mol % of lauryldimethylbenzylammonium chloride and 28 mol % to 32 mol % of myristyldimethylbenzylammonium chloride
d) a mixture comprising 70 mol % of lauryldimethylbenzylammonium chloride and 30 mol % of myristyldimethylbenzylammonium chloride.

14. The antimicrobial mixture according to claim 9, which comprises 4-(3-ethoxy-4-hydroxyphenyl)butan-2-one and the alkyldimethylbenzylammonium salt of formula (II) in amounts such that the 4-(3-ethoxy4-hydroxyphenyl)butan-2-one/salt (II) weight ratio ranges from 5 to 120.

15. A composition comprising, in a physiologically acceptable medium, an antimicrobial mixture according to claim 1.

16. The composition according to claim 15, which comprises at least one additional ingredient chosen from water, oils, polyols containing from 2 to 10 carbon atoms, gelling agents, surfactants, film-forming polymers, dyestuffs, fragrances, fillers, UV-screening agents, plant extracts, cosmetic and dermatological active agents, and salts.

17. The composition according to claim 15, wherein the 4-(3-ethoxy-4-hydroxyphenyl)butan-2-one is present in a content ranging from 0.01% to 5% by weight relative to the total weight of the composition.

18. A non-therapeutic cosmetic treatment process for caring for and/or making up and/or cleansing keratin materials, comprising the application to said keratin materials of a composition according to claim 15.

19. A process for conserving a composition comprising a physiologically acceptable medium which incorporating into said composition an antimicrobial mixture as defined in claim 1.

20. (canceled)

21. A non-therapeutic cosmetic treatment process for caring for and/or making up and/or cleansing keratin materials, comprising the application to said keratin materials of a composition according to claim 16.
